(19) United States
(12) Patent Application Publication
(10) Pub. No.: US 2013/0189772 A1
(43) Pub. Date: Jul. 25, 2013

(54) MULTIWELL-PLATE REACTOR AND SYSTEM THEREFOR

(71) Applicant: Seemab Shaikh, Lebanon, NH (US)

(72) Inventor: Seemab Shaikh, Lebanon, NH (US)

(21) Appl. No.: 13/696,488

(22) PCT Filed: Apr. 25, 2011
(86) PCT No.: PCT/US11/33749

§ 371 Date: Mar. 12, 2013

Related U.S. Application Data
(60) Provisional application No. 61/329,983, filed on Apr. 30, 2010.

(51) Int. Cl. C12M 1/34

(52) U.S. Cl. CPC C12M 41/26; C12M 41/12
USPC 435/297.1

(57) ABSTRACT

A multiwell-plate reactor is provided herein for small scale cell culturing and fermenting. Advantageously, with the multiwell-plate reactor of the subject invention, a small scale cell culturing and fermenting process can be conducted with the fluid, such as a liquid nutrient or one or more gases, being introduced in a controlled manner.

This application claims the benefit of U.S. provisional patent Application No. 61/329,983, filed Apr. 30, 2010, which is herein incorporated by reference in its entirety.

### FIELD OF THE INVENTON

This invention relates to small scale fermentation and cell culture equipment.

### BACKGROUND OF THE INVENTON

Cell culturing and fermentation are known in the prior art for various applications. Large scale fermentors, used for industrial level production, are complex and required to continuously balance the parameters of the fermenting media, such parameters including nutrient levels, oxygen levels, temperature and pH. The fermenting media are also typically agitated, such as by stirring.

Laboratory analysis and other testing may require cell fermentation, but on a small scale. There are, however, limited options for available equipment to handle small scale fermentation with the same parameter controls as a large scale fermentor. One such small scale fermentor is embodied in U.S. Pat. No. 7,374,725 to Klein et al. Klein et al. provide a well plate reactor which includes a plurality of wells for containing cell media. The bottoms of the wells are provided with sensors and gas inlet ports. The sensors provide for monitoring of oxygen levels, pH levels and temperature. Gas flow from the bottoms of the wells not only requires specially modified wells to accept the gas inlets, but, also, gas distribution may be uneven in the media.

In addition, the Klein et al. device does not provide for automated adding of feed. Feed can be added manually from above the samples. For example, methanol or glycerol may be added during the fermentation of Pichia pastoris. The manual feed must be carefully done at controlled amounts. A high oxygen transfer rate into the wells, in combination with the introduction of the feed, may result in high uptake rates of the feed and possible cell lysis.

Thus, there is a need for microreactor systems that provide for precise control of oxygen levels (i.e. gassing), pH levels, feed, and temperature.

### SUMMARY OF THE INVENTION

A multiwell-plate reactor is provided herein for small scale cell culturing and fermenting. The multiwell-plate reactor includes a well plate defining a plurality of wells, each well having an open first end and a second closed end with a sidewall extending therebetween. The multiwell-plate reactor also includes a plate cover emplaceable on the well plate, wherein fluid tight seals are defined between adjacent pairs of the wells with the plate cover emplaced on the well plate. A plurality of inlet ports are mounted to the plate cover, each inlet port being configured to extend into one of the wells through the open first end of the respective well. A supply line extends from each inlet port for receiving a fluid to be introduced into the respective well via the inlet port. Advantageously, with the multiwell-plate reactor of the subject invention, a small scale cell culturing and fermenting process can be conducted with the fluid, such as a liquid nutrient or one or more gases, being introduced through the plate cover. This allows for a more even distribution of the introduced fluid (e.g. liquid and/or gas) into the cell culture media accommodated in the wells.

These and other features of the invention will be better understood through a study of the following detailed description and accompanying drawings.

### BRIEF DESCRIPTION OF THE DRAWINGS

FIG. 1 is a schematic of a system formed in accordance with the subject invention where the fluid is liquid.

FIG. 2 is a schematic of possible exit port locations.

FIG. 3 is a schematic of a system formed in accordance with the subject invention where the fluid is gas.

FIGS. 4A-4B show dissolved oxygen (DO) profiles resulting from prior art technique of manually introducing methanol during induction phase in alternating doses of 50 μl and 125 μl over time.

FIG. 5A shows a dissolved oxygen (DO) profile obtained in a large scale fermentor.

FIG. 5B shows observed experimental dissolved oxygen (DO) profile based on dispensing ranging from 1 μl to about 2.67 μl of 50% methanol every approximately 1-6.2 minutes in series to wells containing 5 ml of cell broth.

FIG. 6 shows an exemplary microreactor set-up utilizing a hold-down fixture, as utilized in Example 2.

FIG. 7 shows a system formed in accordance with the subject invention where gas and liquid are simultaneously delivered.

FIG. 8A shows a configuration of an inlet port useable with the subject invention.

FIG. 8B is a cross-sectional view taken along line 8B-8B of FIG. 8A.

FIG. 9 shows a dissolved oxygen (DO) and pH profiles following bolus feed injection from a Pichia pastoris growth study.

### DETAILED DESCRIPTION OF THE INVENTION

A multiwell-plate reactor is provided herein for small scale cell culturing and fermenting. The multiwell-plate reactor includes a well plate defining a plurality of wells, each well having an open first end and a second closed end with a sidewall extending therebetween. The multiwell-plate reactor also includes a plate cover emplaceable on the well plate, wherein fluid tight seals are defined between adjacent pairs of the wells with the plate cover emplaced on the well plate. A plurality of inlet ports are mounted to the plate cover, each inlet port being configured to extend into one of the wells through the open first end of the respective well. A supply line extends from each inlet port for receiving a fluid to be introduced into the respective well via the inlet port. Advantageously, with the multiwell-plate reactor of the subject invention, a small scale cell culturing and fermenting process can be conducted with the fluid, such as a liquid nutrient or one or more gases, being introduced through the plate cover, or through a combination of through the cover at the top. It is noted that gas delivery through the top is preferred; however, the reactor can be configured for gas delivery from the bottom in situations where this would be beneficial, while still providing the option for delivery of liquid through the top. The present system allows for a more even distribution of the introduced fluid (e.g. liquid and/or gas) into the cell culture media and more even growth of cells across the wells.

With reference to the Figures, a multiwell-plate reactor 10 is shown and described herein. The reactor 10 includes a well plate 12, defining a plurality of wells 14; a plate cover 16 emplaceable on the well plate 12; a plurality of inlet ports 18 mounted to the plate cover 16; and, a supply line 20 extending from each of the inlet ports 18 for receiving a fluid to be introduced into the wells 14 via the inlet ports 18. The reactor 10 permits various applications, including automated introduction of feed in the form of liquid nutrient and control of gas flow for small scale cell culturing and fermenting.

The wells 14 are each formed with an open first end 22, a closed second end 24 and a side wall 26 extending therebetween. The well plate 12 may be provided with any number of the wells 14 configured in various arrays, such as a 24-well configuration in a 4×6 array. It is preferred that the wells 14 be sized to accommodate samples in the range of 3-7 ml. As will be appreciated by those skilled in the art, any configuration of the wells 14 consistent with the subject invention may be utilized.

The well plate 12 may be formed of any material generally stable for the cell culturing and fermentation process. Preferably, the well plate 12 is formed of a thermoplastic, such as polystyrene. Other materials may be suitable, such as glass. In addition, the interior surfaces of the wells, such as the interior surfaces of the closed second ends 24 and/or the side walls 26, may be coated or treated to enhance or diminish surface binding of the cell media. In this manner, target areas may be pre-defined for binding. In addition, differentiation of cells, where a concern exists, may be minimized.

The plate cover 16 is preferably formed to be at least coextensive with the upper surface of the well plate 12 so as to at least completely cover all of the open first ends 22 of the wells 14. The plate cover 16 is preferably formed to define fluid-tight seals between adjacent pairs of the wells 14 with the plate cover 16 being emplaced on the well plate 12. The seals may be defined by direct interaction between the well plate 12 and the plate cover 16 such as through sufficient spacing between the open first ends 22 of the wells 14; sufficiently tight interfaces between the well plate 12 and the plate cover 16 at locations between adjacent open first ends 22; and/or, sufficiently tortuous paths being defined at the interfaces of the well plate 12 and the plate cover 16 at locations between adjacent open first ends 22. In addition, or alternatively, a sealing material, such as an elastomeric gasket or vent member(s) (as described below), may be disposed between the well plate 12 and the plate cover 16 which extends between adjacent open first ends 22.

As will be appreciated by those skilled in the art, a plurality of plate covers 16 may be utilized, each sized to cover one or more of the open first ends 22 of the wells 14. Each of the wells 14 may be covered by an individual plate cover 16 or a plate cover 16 may be provided for covering a sub-set of the wells 14.

To maintain the fluid-tight seals, it is preferred that the plate cover 16 be releasably retained on the well plate 12. The releasable retaining arrangement may include cooperating mechanical elements (such as a snap detent/recess combination); external holding members (such as a hold-down jig or fixture for applying pressure to the plate cover 16 on the well plate 12); applied vacuum; and/or releasable adhesive or chemical bond. In any regard, it is preferred that the plate cover 16 be retained with sufficient force on the well plate 12 to maintain fluid tight seals between the wells 14, with the plate cover 16 being removable to provide access to the cell media contained within the wells 14 as needed.

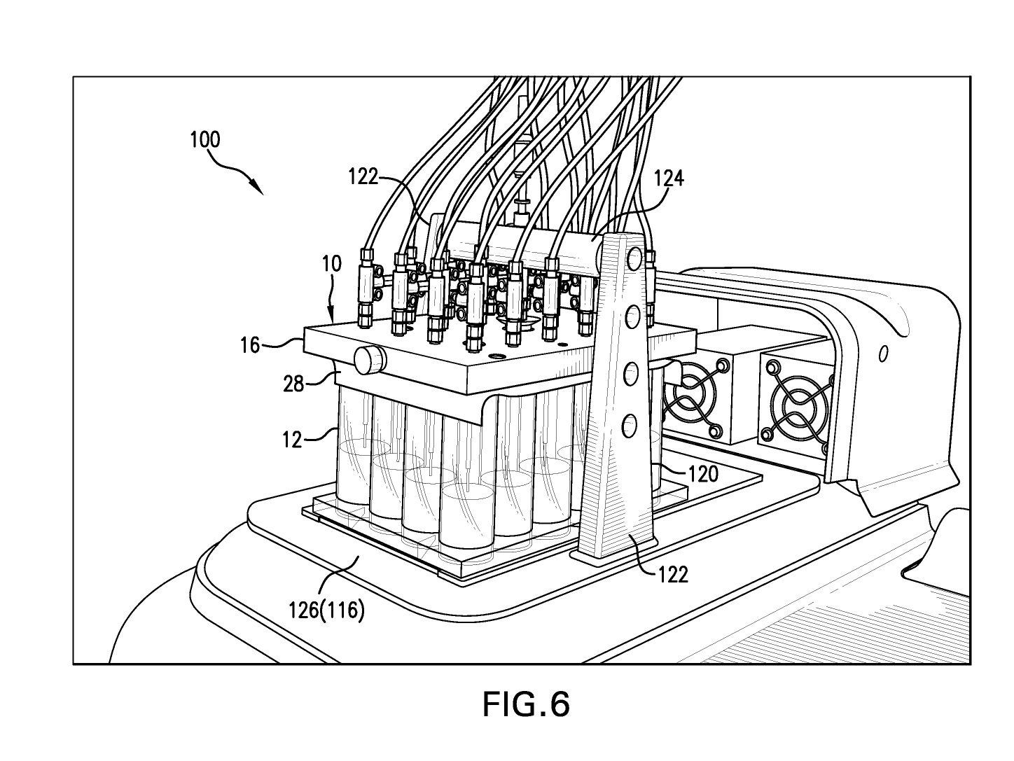

FIG. 6 shows an exemplary hold-down fixture 120 useable with the subject invention. The hold-down fixture 120 includes at least one, preferably two, stanchions 122 located adjacent to the reactor 10. An engagement arm 124 extends transversely from at least one of the stanchions 122 configured and located to be in pressing engagement with portions of the reactor 10 during use. With the reactor 10 resting on base 126, the engagement arm 124 is configured and located to apply a pressing force against the reactor 10, particularly so as to press the plate cover 16 down onto the well plate 12 in defining a seal therebetween. The engagement arm 124 may be detachably or pivotably connected to the at least one stanchion 122 so as to be movable in providing access to the reactor 10 as needed. Preferably, the engagement arm 124 extends between two of the stanchions 122, with the stanchions 122 being located on opposite sides of the reactor 10 and the engagement arm 124 spanning the reactor 10.

As will be appreciated by those skilled in the art, sufficient distribution of the pressing force generated by the hold-down fixture 122 is desired so as to ensure sealing for all of the wells 14. Points of interengagement between the engagement arm 124 and the reactor 10 (e.g., locations, area of contact) and size of pressing force should be considered. In addition, more than one of the engagement arms 124 may be utilized extending from each of the stanchions 122. With a plurality of the engagement arms 124, the engagement arms 124 may be spaced apart (e.g., in a v-configuration) so as to distribute the pressing force.

Maintenance of the seals during use is important. Where the reactor 10 is placed atop an agitator 116, any sealing arrangement is configured to maintain the seals during agitation of the reactor 10. The hold-down fixture 120 may be located on the agitator 116 with the reactor 10 so as to move in concert with the reactor 10.

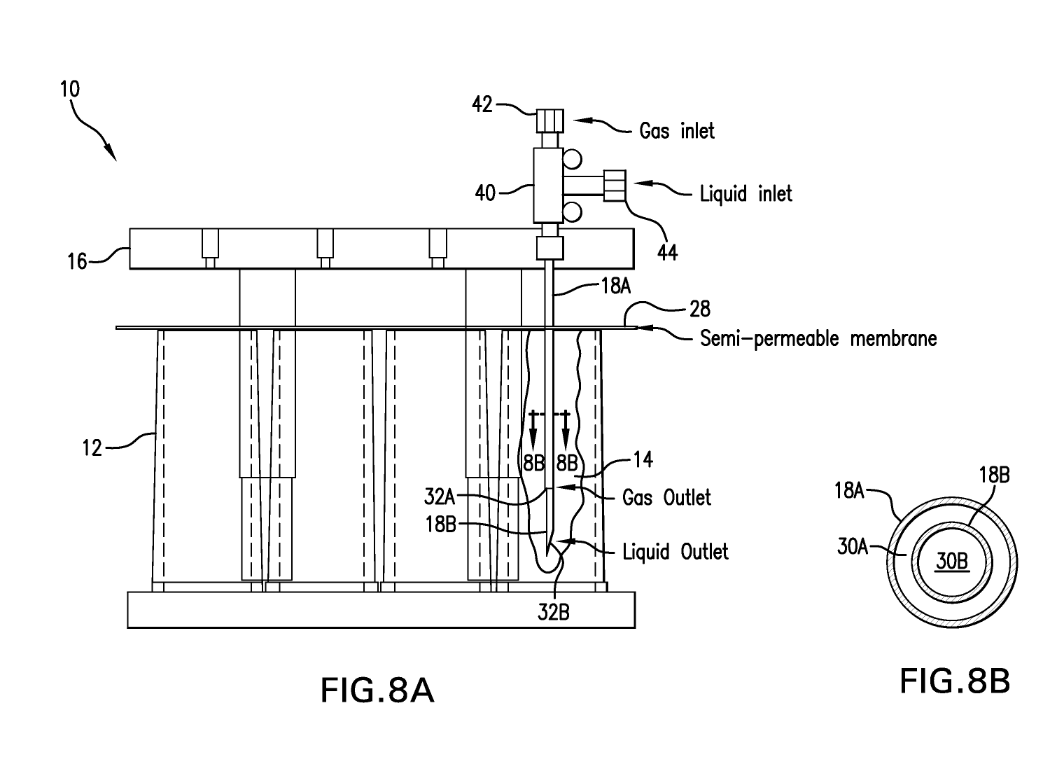

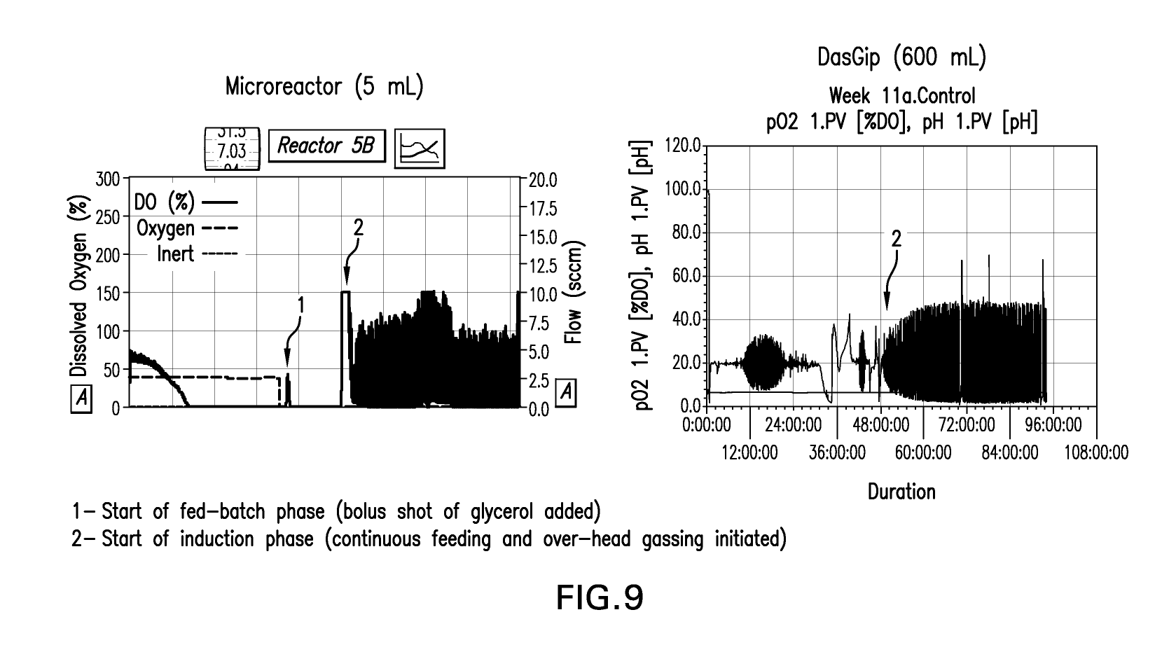

It is preferred to provide venting for each of the wells 14. Preferably, a vent member 28 is located in communication with each of the wells 14. To permit proper venting, one or more vent members 28 may be provided in the plate cover 16 in communication with the wells 14. Openings 27 may be formed in the plate cover 16 in communication with the vent members 28 such that venting is achieved through the plate cover 16. The vent members 28 may be individually seated in each of the openings 27. The vent members 28 may be sized smaller than the diameters of the respective open first ends 22 and located to be wholly above the respective open first ends 22 (i.e., not located to extend beyond any portion of the open first ends 22). Alternatively, as shown in FIGS. 6 and 8A, one or more of the vent members 28, in the form of a film or mat, may be interposed between the well plate 12 and the plate cover 16 so as to extend across all or a sub-set of the wells 14. Here, the openings 27 need not be provided as venting may be achieved through side edges of the vent members 28 between the well plate 12 and the plate cover 16. Any semi-permeable membrane having a pore size of 0.22 microns or smaller may be utilized for the vent members 28 which preferably permits gas transmission therethrough, but not microbial transmission. Polytetrafluoroethylene (PTFE) may be utilized as the vent material.

The inlet ports 18 extend from the plate cover 16 into each of the wells 14 through the respective open first end 22. As such, the inlet ports 18 are introduced into the wells 14 from above any samples contained in the wells 14. As shown in FIG. 8A, the inlet ports 18 may extend through the vent member(s) 28, particularly if the vent member(s) 28 are interposed between the well plate 12 and the plate cover 16. Preferably, the vent member(s) 28 sealingly engage about the inlet ports 18. One or more of the inlet ports 18 may extend into each of the wells 14 so as to permit simultaneous introduction of a plurality of fluids.

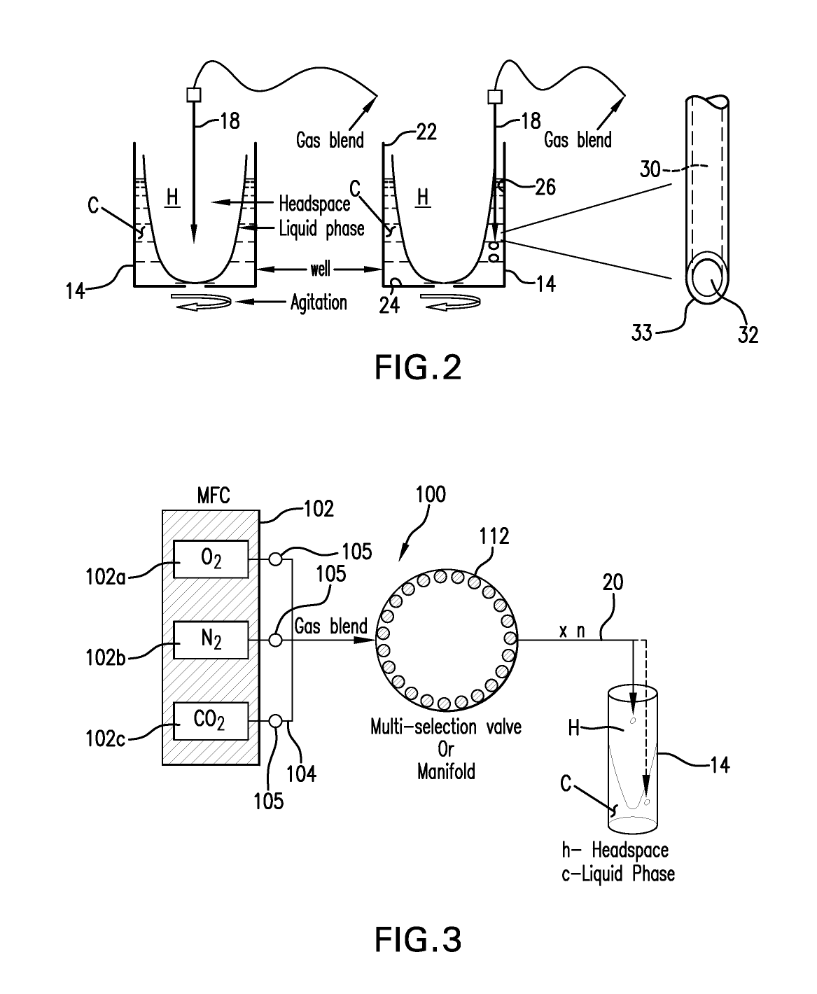

The inlet ports 18 each include a fluid path 30 defined therethrough which terminates at an exit port 32. By way of non-limiting example, the inlet ports 18 may include a needle cannula having a lumen which defines a portion of the fluid path 30. With reference to FIG. 2, the inlet ports 18 may be configured to have the exit ports 32 located in a head space H above any cell media C contained in the wells 14 or located to be submersed within the cell media C. It is preferred that the exit ports 32 be located in proximity to the sidewalls 26, particularly where stirring or other agitation is utilized on the cell media C resulting in wave flow of the cell media C about the interior surface of the wells 14, particularly the side walls 26.

Location of the exit ports 32 affects the manner in which the fluid is delivered to the cell media C. With the exit ports 32 in the head space H, fluid is introduced above the cell media C. This may be desired where the fluid is not volatile. With the exit ports 32 submersed in the cell media C, fluid is delivered directly into the cell media C. This may be desired where the fluid is volatile such as methanol. Submersed delivery avoids evaporation. Also, the exit ports 32 may be defined on beveled ends 33 of the inlet ports 18 (FIG. 2), having one or more bevels.

The inlet ports 18 are preferably fixed to the plate cover 16 so as to be removable therewith. It is preferred that the inlet ports 18 be fixed to rigid portions of the plate cover 16, e.g., spaced from the openings 27, so as to minimize any relative movement therebetween.

The supply lines 20 are preferably provided in a one-to-one correspondence to the inlet ports 18 so that individual flow to each of the inlet ports 18 may be separately controlled. It is preferred that the supply lines 20 be flexible, and formed with sufficient length to have slack during use. The supply lines 20 may be formed of a polymeric material, such as PVC. Stability of the material of the supply lines is required, particularly since fluid may be contained therein for varying intervals of time, depending on rate of introduction of the fluid.

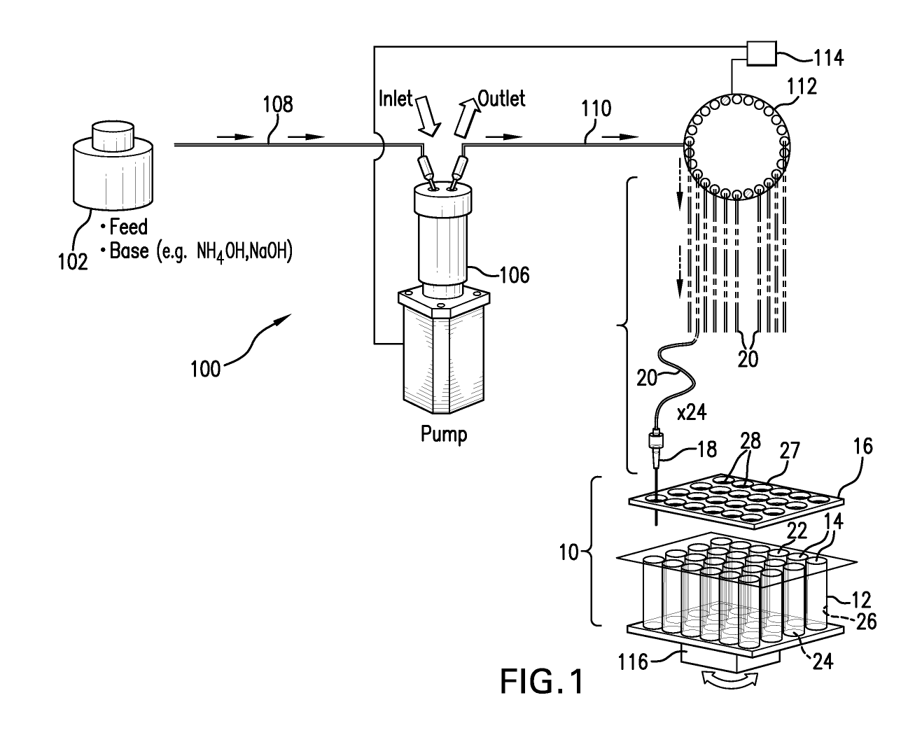

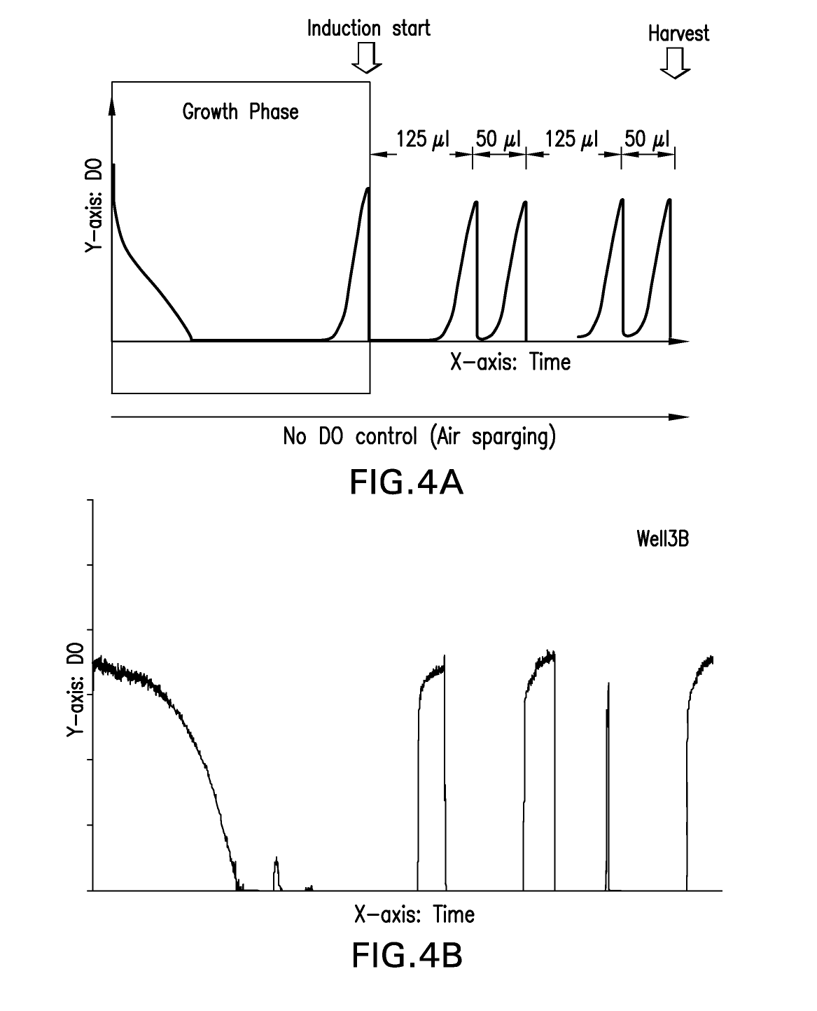

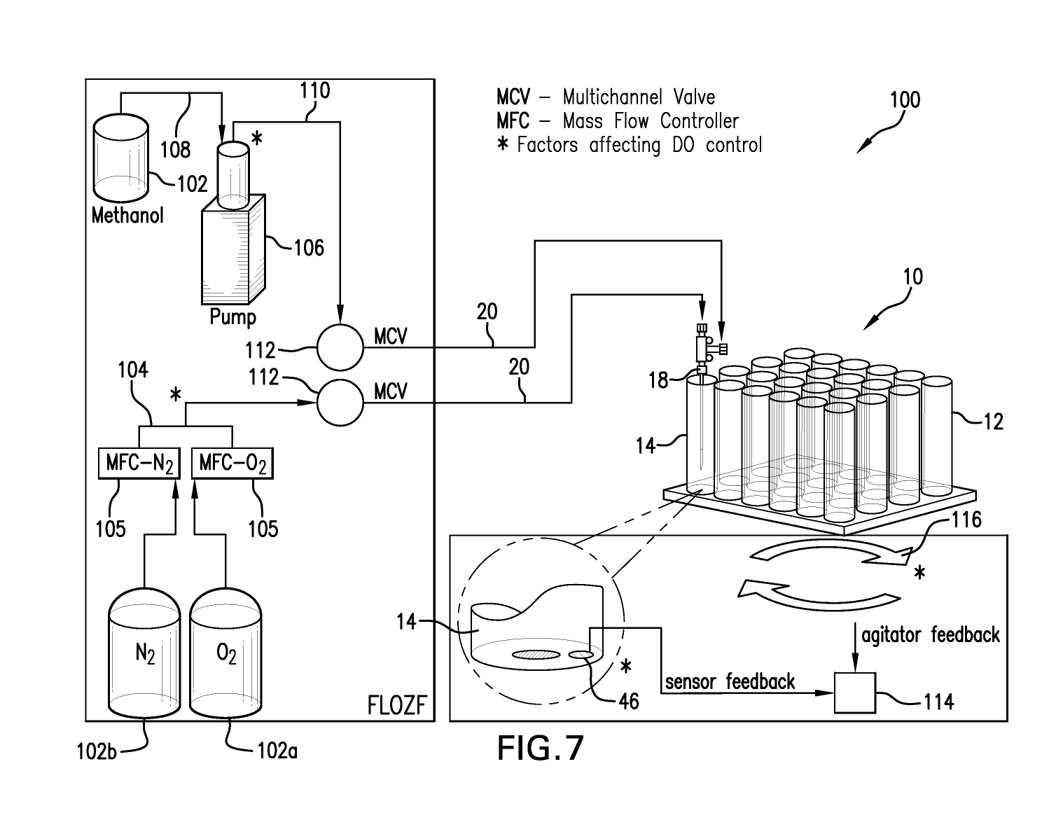

The multiwell-plate reactor 10 may be used in a system 100 to conduct cell culturing and fermenting. The system 100 includes at least one source of fluid 102. The source of fluid 102 may be a source of liquid nutrient, such as methanol or glycerol or combination thereof (FIG. 1). It is noted that even viscous liquids such as glycerol are amenable to controlled delivery into the wells in the present system. As will be appreciated by those skilled in the art, other forms of liquids, including other liquid nutrients, may be utilized. For example, the liquid may be, or include, a base, such as sodium hydroxide (NaOH) and/or ammonium hydroxide (NH₄OH), for introduction into the wells 14 for regulating the pH of the cell media C. In addition, or alternatively, the source of fluid 102 may be one or more gases including, but not limited to, oxygen, nitrogen and/or carbon dioxide (FIG. 3). The source of fluid 102 may have pre-mixed components therein or, as shown in FIG. 3, include a plurality of separate reservoirs 102a, 102b, 102c, etc. which are caused to be mixed within the system 100, e.g., by a manifold 104 optionally regulated by one or more mass flow controllers 105. The reservoirs 102a, 102b, 102c may contain individual constituent gases, or blends. The gases may be blended, such as blended nitrogen and oxygen, using any known technique. The system 100 may be also configured to deliver simultaneously both liquid nutrient and gas (FIG. 7).

With the fluid being a liquid, one or more pumps 106 (FIG. 1) may be provided to urge the liquid from the source of fluid 102 as needed. One or more suction lines 108 may be provided in communication with the sources of fluid 102 which communicate with the suction side of the pumps 106. One or more discharge lines 110 may extend from the discharge side of the pumps 106. The supply lines 20 may serve as the discharge lines 110.

With respect to the fluid being one or more gases, the gases may be pressurized within the source of fluid 102. In addition, or alternatively, a compressor or other pressure-inducing element may be provided to urge the gases from the sources of fluid 102 into the supply lines 20 as needed. It is noted that gas delivery through the plate cover 16, as described above, is preferred; however, the reactor 10 can be configured for some or all gas delivery from the closed second ends 24 of the wells 14 in situations where this would be beneficial.

Multiple sources of fluid 102 may be provided in communication with each of the supply lines 20. Preferably, at least one control valve 112 is provided to regulate flow from the sources of fluid 102 to the supply lines 20. A controller 114 may be provided to control the at least one control valve 112. The controller 114 may include a programmable CPU to accept commands for controlling flow rate through the at least one control valve 112. Preferably, a single control valve 112 is provided to permit selective communication between one of the sources of fluid 102 and the wells 14, via the supply lines 20, although a control valve 112 may be provided for any subset of the supply lines 20.

Preferably, the control valve 112 is configured to permit communication between the source of fluid 102 and a single of the supply lines 20 at an instance. The control valve 112 may be an adjustable multi-port valve to permit such selective communication. The control valve 112 is preferably adjustable to allow selective communication of the supply lines 20 with the source of fluid 102 in series. In this manner, fluid may be introduced into each of the wells 14 in intervals. The intervals, which may be equal or varied, may be adjusted and programmed into the controller 114.

One or more of the pumps 106, as well as any pressure-inducing element for the gasses, may be operatively coupled to the controller 114 to be controlled thereby. The controller 114 may control the rate of fluid introduction by controlling the pumping rate. It is preferred that the pump 106 be caused to de-activate between successive dosings, thereby permitting the control valve 112 to adjust without any pumping. It is further preferred that dosing occur with the pump 106 being activated, then deactivated, with a time interval lapsing after deactivation before the control valve 112 is adjusted. It is further preferred to allow a time interval to lapse after adjustment of the control valve 112 and before the pump 106 is activated for dosing. Time intervals may also be introduced during feed cycles so as to cause delay between cycles.

The reactor 10 may be mounted to the agitator 116 which may cause stirring or other agitation of the media in the wells 14. The flexibility of the supply lines 20 provides continuous connections for fluid to the reactor 10 as it moves.

In a preferred arrangement, as shown in FIG. 7, each of the wells 14 is provided with two of the inlet ports 18: one for introducing one or more gases, and the other for introducing liquid nutrient. This arrangement results is parallel set-ups in that separate supply lines 20, sources of fluid 102, control valves 112 and controllers 114 are provided for the one or more gases and, separately, for the liquid nutrient.

With reference to FIG. 8A, two or more of the inlet ports 18 may be formed unitarily where multiple fluid paths 30 are arranged coaxially. As shown in FIG. 8A, a first inlet port 18A may be located coaxially about a second inlet port 18B. Preferably, the second inlet port 18B extends through and beyond exit port 32A of the first inlet port 18A. In this manner, separate fluid paths 30A, 30B are provided with the exit ports 32A, 32B of the first and second inlet ports 18A,18 being spaced apart. It is further preferred that the second inlet port 18B have a smaller outer diameter than the inner diameter of the first inlet port 18A such that the fluid path 30A of the first inlet port 18A is at least partially defined by the annulus defined between the first and second inlet ports 18A, 18B (FIG. 8B).

It is preferred that the second inlet port 18B be utilized to introduce the liquid component and that the first inlet port 18A be utilized to introduce the one or more gases. Liquid introduced through the second inlet port 18B will be spaced from, and located gravitationally below, the exit port 32A of the first inlet port 18A. In this manner, blockage of the exit port 32A of the first inlet port 18A may be avoided, even with liquid collecting at the exit port 32B of the second inlet port 18B due to capillary attraction or residual build-up.

As shown in FIG. 8A, the first and second inlet ports 18A, 18B may extend from a common body 40 having both a gas inlet 42 and a liquid inlet 44. The gas and liquid inlets 42, 44 are formed separate. The fluid path 30B is in closed communication with the liquid inlet 44, and the fluid path 30A is in closed communication with the gas inlet 42.

In operation, the reactor 10 is initially assembled with the cell media C of interest being disposed in the wells 14. With the cover plate 16 emplaced on the well plate 12, the supply lines 20 are caused to be primed by the fluid. With the supply lines 20 being fully primed, further introduction of fluid into the supply lines 20 causes displacement therefrom of a dose of liquid nutrient or gas, depending on the fluid. The amount of fluid being dosed, e.g., liquid nutrient, may be a function of the volume of the wells 14 (e.g., total target liquid nutrient being 2%-5% of the total working volume of the wells 14) and/or volume per unit time (constant or varying) (e.g., gas feed rate of 0.5 ml/min to 20 ml/min. The system 100 may become a positive displacement system in that the amount of fluid introduced in a supply line 20 will result in a corresponding amount of fluid being dispensed through the corresponding inlet port 18. Very low pressures and doses are envisioned for use with the system 100. Accordingly, uncontrolled discharge through the inlet ports 18 is not expected. Check valving may be utilized if necessary to minimize uncontrolled flow.

The reactor 10 is preferably formed to have its various components be sterilizable, e.g., by autoclaving or gamma or gas (e.g., EtO) sterilization. Components of the system 100 may need to be maintained in a clean environment with the reactor 10 and, thus, may need to be sterilizable. It is preferred that the pumps 106 be sterilizable, e.g., by autoclaving.

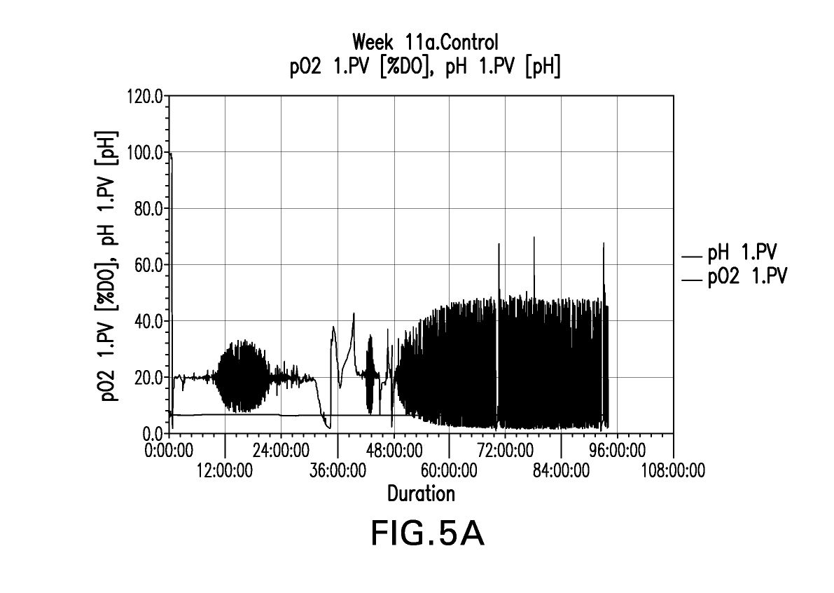

With the subject invention, liquid nutrient can be introduced in an automated and controlled manner into the wells 14. FIGS. 4A and 4B depict the technique of manually introducing feed (methanol) into a microreactor in alternating doses of 50 μl and 125 μl over time. In the absence of a continuous feeding system the microreactor requires bolus additions of carbon source (Methanol). As a result, the process carried out in the microreactor is changed from a typical Excess Dissolved Oxygen (“DO”)—Limited Methanol process (present in large scale fermentors) to a Limited DO and Excess Methanol process. In particular, instead of controlling the DO at a given set point (e.g. 20%) and feeding methanol at a very slow flow rate (as it is typically done in large scale fermentors), a DO limited environment is created in the microreactor such that the Methanol Consumption Rate (MCR) of the culture remains approximately the same as larger fermentors. The DO spike in FIG. 4B is observed when the cells finish consuming all the available carbon source indicating addition of the next bolus methanol shot.

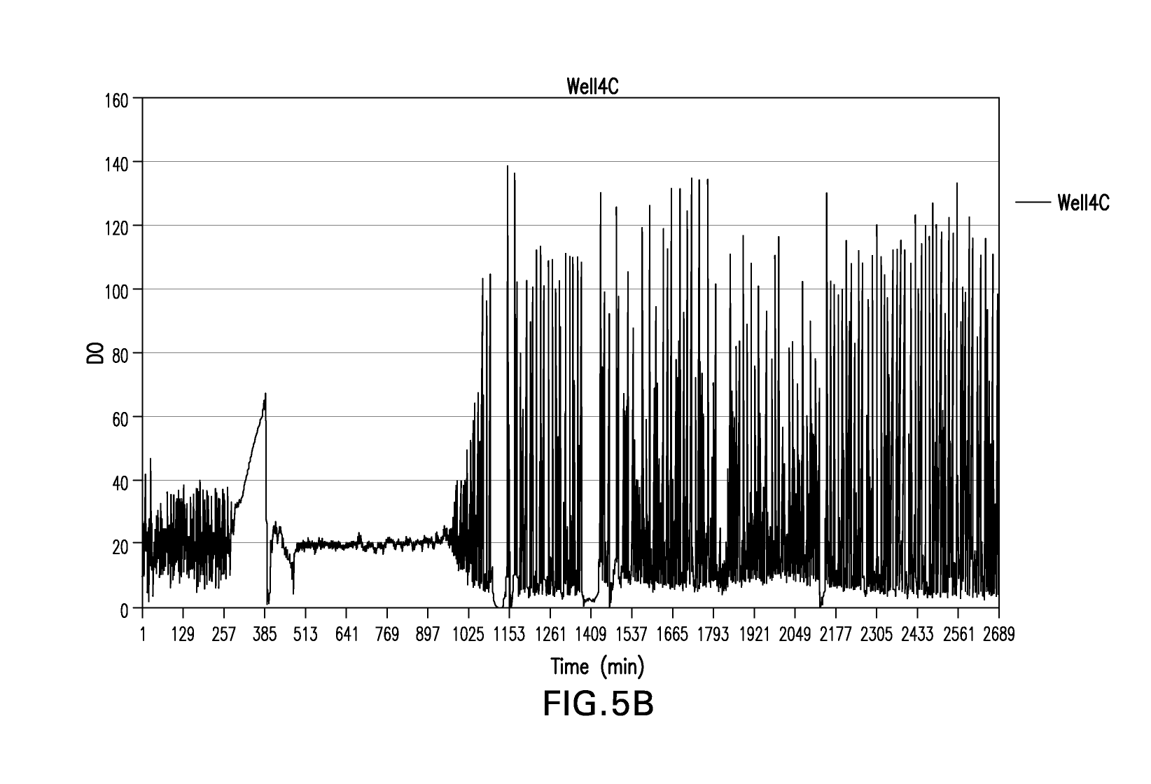

With reference to FIGS. 5A-5B, controlled automated introduction of feed, as obtainable by the system 100, results in DO profiles similar to those observed in larger fermentors running with Excess Oxygen-Limited Methanol process. FIG. 5A depicts a dissolved oxygen (DO) profile obtained in a 0.5 L fermentor. FIG. 5 depicts an observed experiment dissolved oxygen (DO) profile based on dispensing a range of about 1 μl to about 2.67 μl of 50% methanol every about 1-6.2 minutes in series to wells containing 5 ml of cell broth. In larger fermentors, as depicted in FIG. 5A, the dissolved oxygen is maintained close to the set point of 20%. The spikes and dips in dissolved oxygen (DO) observed in the DO profiles indicate a slow but continuous methanol feeding.

To aid in the automated supply of gas and/or liquid, one or more sensors 46 may be located in each of the wells 14 (FIG. 7) configured to monitor different parameters in the cell media, such as gas levels (e.g. oxygen, CO₂, etc.), metabolites or nutritional supplements (e.g. glycerol, methanol, etc.), or optical density parameters (e.g. OD₆₀₀, etc.) Any combination of different parameters can be monitored. Information collected by the sensors 46 may be transmitted to the controller 114. The controller 114 may be configured to responsively adjust one or more of the inputs into the reactor 10 depending on detected levels. In addition, the controller 114 may be operatively connected to the agitator 116 to control operation thereof.

### EXAMPLE 1

A pump having a flow rate of 6 nl/min-10 ml/min may be utilized, such as a positive displacement pump sold under the trademark “milliGAT” by Global FIA, Inc. of Fox Island, Wash. The control valve may be a multiport valve, such as a stream selector valve, sold under the model number C35Z by Valco Instruments Co. Inc. of Houston, Tex. (having 20-26 ports). A suction line may extend from a reservoir of liquid nutrient (e.g., methanol) to the suction inlet of the pump, with one discharge line extending from the discharge outlet of the pump to a main inlet of the control valve. Supply lines extend from each of the outlet ports of the control valve to the inlet ports of the multiwell-plate reactor. The suction line, discharge line and supply lines may be each formed from PVC tubing, e.g., 1/16″ HPLC tubing. The inlet ports may include a 20 gauge needle cannula connected to the supply lines by luer-lock connectors in an arrangement such as in FIG. 1.

### EXAMPLE 2

Pichia Pastoris Growth Study

TABLE 1
BMGY Media for Pichia pastoris Growth Study

| Component | Quantity/L | |
|---|---|---|
| Hy-Soy (Kerry) | 20 | g/L |
| Yeast Extract | 10 | g/L |
| 1M Potassium Phosphate Buffer, pH 6.5 | | |
| Sorbitol | 18.2 | g/L |
| Glycerol | 40 | g/L |
| 10X YNB w/ Ammonium Sulfate w/o Amino Acids | 13.4 | g/L |
| 250X Biotin (0.4 g/L) | 10 | ml/L |
| 500X Chloramphenicol (50 g/L) | 1 | ml/L |
| 500X Kanamycin (50 g/L) | 2.5 | ml/L |

| | |
|---|---|
| DI Water | — |

A P. pastoris seed culture was grown in a shake flask with 100 ml of 4% BMGY media (stock BMGY media is described in Table 1), at a temperature of about 24° C., with rocking at about 180 rpm, for about 3 days (or through about the end of log phase).

An aliquot of about 0.5 ml of the seed culture was added to about 4.5 ml of 4% MBGYmedia (described in Table 1) forming the experimental/test cell culture, which was added to each well of a multiwell-plate reactor (i.e., each of the 24 wells contained about 5.0 ml of test culture). The multiwell-plate reactor containing the P. pastoris test cultures was incubated for about 8-12 hours under the following conditions: the pH was maintained at about 6.5, the temperature was maintained at about 24° C., the dissolved oxygen level (DO) was maintained at about 20%, and the reactor was set at about 180 rpm to provide rocking/shaking of the cultures. After the about 8-12 hour incubation period a 2.5% glycerol shot was added (i.e., a liquid nutrient feed). After the glycerol shot, the P. pastoris culture was incubated for about 62.5 hours. The Feed pump delivered 50% methanol liquid feed in an amount ranging from about 1 μl to about 2.67 μl, at intervals of about every 1-6.2 minutes, to each well during the incubation period. The liquid feeding and gassing were both delivered through top delivery through the gas outlet and liquid outlet, as depicted in FIGS. 6 and 8A. During the incubation, the pH was maintained at about 6.5, the temperature was maintained at about 24° C., the dissolved oxygen (DO) level was about 20% and the reactor was set at about 180 rpm to provide rocking/shaking of the cultures.

The growth of the P. pastoris test cultures was measured from samples taken from sections across the multiwell-plate reactor. Exemplary values (wet cell weight, WCW in g/L)) ranged from 424 g/L for well 1 (located on the top left end of the multiwell plate), to 464 g/L for well 12 located at the end of the second row of the plate. The average value was ˜420 g/L, with a standard deviation of about 35 g/L and an error of ±7 (several wells were not included in the average due to contamination or set-up error). Values for the corner wells were 436 g/L for well 19 and 439 g/L for well 24.

These results illustrate that P. pastoris cells can be grown with uniform results in a 24-well multiwell-plate reactor system under conditions that replicate large-scale production methods. The P. pastoris test cells showed uniform growth under controlled pH, temperature, and feeding and also illustrate the general utility of this system for eukaryotic cultures, such as yeast, and would be expected to be similarly useful for mammalian cells, such as CHO cells.

### EXAMPLE 3

Escherichia coil Growth Study

TABLE 2

| | E. coli Media | | |
|---|---|---|---|
| 1.10 | Seed Media | | |
| | LB Media | | |
| | Ampicilin | 100 | mg/L |
| | Chloramphenicol | 34 | mg/L |
| 1.11 | 10× Phosphate Buffer with | | |
| | Vitamins & Minerals | | |
| | Potassium Phosphate (Dibasic) | 70 | g/L |
| | Potassium Phosphate | 30 | g/L |
| | (Monobasic) | | |
| | Ammonium Sulfate | 70 | g/L |
| | Magnesium Sulfate | 3 | g/L |
| | Calcium Sulfate or Chloride | 1 | g/L |
| | Nicotinic acid | 0/2 | g/L |
| 1.12 | Production Media | | |
| | LB Media | 792 | mL |
| | Item 1.11 | 8 | ml |
| 1.13 | Feed-Batch Solution | | |
| | 50% Glycerol | | |
| | Yeast Extract | 60 | g/L |
| | Magnesium Sulfate | 2.5 | g/L |

An E. coli seed culture was grown in a 100 ml shake flask at an initial ˜OD 1.10 in the seed media (described in section 1.10 in Table 2), about 37° C., with agitation/rocking at about 180 rpm, for 8-10 hours. An aliquot of about 0.5 ml of the seed culture was added to about 3.5 ml of production media (described in section 1.12 in Table 2) forming the experimental/test cell culture, which was added to each well of a multiwell-plate reactor (i.e., each of the 24 wells contained about 4.0 ml of test culture). The multiwell-plate reactor containing the E. coli test cell cultures was incubated for about 3 hours under the following conditions. The pH was maintained at about 7.0, the temperature was maintained at about 32° C., the dissolved oxygen (DO) level was about 20% and the reactor was set at about 600 rpm to provide agitation, i.e., rocking/shaking of the cultures. The DO level was maintained by gassing from the bottom of the wells. The multiwell-plate reactor system was set for feeding from the top of the wells with the Feed-Batch solution (described in section 1.13 of Table 2), ranging in an amount of from about 1 μl-2.67 μl of Feed Batch solution (1.13) every 1-6.2 minutes. The growth of the E. coli test cultures was measured from samples taken from sections across the multiwell-plate reactor. Exemplary values (wet cell weight (WCW) in g/L) ranged from 41 g/L for well 1 (located on the top left end of the multiwell plate), to 56 g/L for well 15 located near the middle of the plate. The average value was ˜46 g/L (n=19), with a standard deviation of about 6 and an error of ±1 (several wells were not included in the average due to contamination or set-up error). Values for the corner wells were 44 g/L for well 19 and 46 g/L for well 24.

These results illustrate that E. coli cells can be grown with uniform results in a “micro”multiwell-plate reactor system under conditions similar to large-scale production methods. The E. coli test cells showed uniform growth under controlled pH, temperature, gassing, and feeding and also illustrate the general utility of this system for bacterial or prokaryotic cultures, as well as mammalian and other eukaryotic cell culture systems.

What is claimed is:

1. A multiwell-plate reactor comprising:
a well plate defining a plurality of wells, each said well having an open first end and a second closed end with a sidewall extending therebetween;
a plate cover emplaceable on said well plate, wherein fluid tight seals are defined between adjacent pairs of said wells with said plate cover emplaced on said well plate;
a plurality of inlet ports mounted to said plate cover, each said inlet port being configured to extend into one of said wells through said open first end of respective said well; and,
a supply line extending from each said inlet port for receiving a fluid to be introduced into respective said well via said inlet port.

2. A multiwell-plate reactor as in claim 1, wherein said inlet port extends into respective said well in proximity to said sidewall of respective said well.

3. A multiwell-plate reactor as in claim 1, wherein said inlet port terminates in proximity to said second closed end of respective said well.

4. A system for cell fermenting, said system comprising:
a multiwell-plate reactor as in claim 1; and
at least one source of fluid in communication with said supply lines.

5. A system as in claim 4, wherein said fluid is a liquid nutrient.

6. A system as in claim 5, further comprising at least one pump for urging said liquid nutrient from said source.

7. A system as in claim 5, further comprising a control valve for selectively permitting communication between said source of liquid nutrient and a plurality of said supply lines.

8. A system as in claim 7, wherein said control valve is adjustable to permit communication between a single of said supply lines and said source of liquid nutrient at an instance.

9. A system as in claim 4, wherein said fluid is at least one gas.

10. A system as in claim 9, further comprising at least one control valve for controlling flow of said at least one gas.

11. A system as in claim 9, further comprising a control valve for selectively permitting communication between said source of at least one gas and a plurality of said supply lines.

12. A system as in claim 11, wherein said control valve is adjustable to permit communication between a single of said supply lines and said source of at least one gas at an instance.

13. A system as in claim 11, further comprising at least one stanchion disposed adjacent to said multiwell-plate reactor with at least one engagement arm extending therefrom configured and located to be in pressing engagement with portions of said multiwell-plate reactor.

14. A multiwell-plate reactor system comprising:
a well plate defining a plurality of wells, each said well having an open first end and a second closed end with a sidewall extending therebetween;
a plate cover emplaceable on said well plate, wherein fluid tight seals are defined between adjacent pairs of said wells with said plate cover emplaced on said well plate;
a plurality of gas inlet ports mounted to said plate cover, each said gas inlet port being configured to extend into one of said wells through said open first end of respective said well;
a source of at least one gas;
a gas supply line extending from each said gas inlet port for receiving gas from said source of at least one gas to be introduced into respective said well via said gas inlet port;
a plurality of liquid inlet ports mounted to said plate cover, each said liquid inlet port being configured to extend into one of said wells through said open first end of respective said well;
a source of liquid nutrient; and,
a liquid supply line extending from each said liquid inlet port for receiving liquid from said source of liquid nutrient to be introduced into respective said well via said liquid inlet port.

15. A multiwell-plate reactor system as in claim 14, further comprising a control valve for selectively permitting communication between said source of liquid nutrient and a plurality of said liquid supply lines.

16. A multiwell-plate reactor system as in claim 15, wherein said control valve is adjustable to permit communication between a single of said liquid supply lines and said source of liquid nutrient at an instance.

17. A multiwell-plate reactor system as in claim 14, further comprising at least one control valve for selectively permitting communication between said source of at least one gas and a plurality of said gas supply lines.

18. A multiwell-plate reactor system as in claim 17, wherein said control valve is adjustable to permit communication between a single of said gas supply lines and said source of at least one gas at an instance.

19. A multiwell-plate reactor system as in claim 14, further comprising at least one stanchion disposed adjacent to said multiwell-plate reactor with at least one engagement arm extending therefrom configured and located to be in pressing engagement with portions of said multiwell-plate reactor.
